(19) United States
(12) Patent Application Publication
(10) Pub. No.: US 2025/0241828 A1
(43) Pub. Date: Jul. 31, 2025

(54) SYSTEM, APPARATUS, AND METHOD FOR RELIEVING ABDOMINAL DISTENTION

(71) Applicant: AVENT, INC., Alpharetta, GA (US)

(72) Inventors: Emily POSEY, Alpharetta, GA (US); Madeline WARTAN, Alpharetta, GA (US); Mariann CARY, Alpharetta, GA (US); Donald J. MCMICHAEL, Alpharetta, GA (US); Anthony D. ROBERTS, Alpharetta, GA (US); Tyler CROWE, Alpharetta, GA (US); Lesley KENT, Alpharetta, GA (US)

(21) Appl. No.: 19/111,147

(22) PCT Filed: Sep. 12, 2023
(86) PCT No.: PCT/US2023/032465

§ 371 Date: Mar. 12, 2025

Related U.S. Application Data
(60) Provisional application No. 63/405,555, filed on Sep. 12, 2022.

(51) Int. Cl. A61J 15/00; A61M 1/00

(52) U.S. Cl. CPC A61J 15/0096; A61J 15/0015; A61M 1/67; A61M 2205/7536; A61M 2209/08; A61M 2210/1053; A61M 2240/00

(57) ABSTRACT

A gastric decompression apparatus includes a variable-volume container having a movable plunger. The movable plunger is slidably mounted within the containment barrel so as to vary the contained volume of the container. The movable plunger has a plunger body having at least one lumen extending therethrough, and a filter disposed within the lumen. The gastric decompression apparatus may have a larger diameter and a shorter length from the first end to the second end as compared to conventional elongated syringes having an equal volume to minimize the size of apparatus within an isolette for a neonatal patient, advantageously reducing crowding within the isolette, particularly when there may be numerous tubes, wires, and monitors present for treating the neonatal patient. A gastric decompression system includes the gastric decompression apparatus and a catheter coupled to the container. A method of providing gastric decompression using the gastric decompression system is also provided.

### RELATED APPLICATIONS

The present application claims priority to U.S. Provisional Application Ser. No. 63/405,555, filed on Sep. 12, 2022, which is incorporated herein in its entirety by reference thereto.

### FIELD OF THE INVENTION

The subject matter of the present invention relates generally to systems, apparatus and methods for relieving gastric pressure and/or abdominal distention during enteral feeding.

### BACKGROUND

The Neonatal Intensive Care Unit (NICU) is a hospital nursery where infants receive specialized care as a result of preterm birth defined as less than 37 weeks of gestation, low birth weight, or health conditions that require continuous care. Out of the 140 million infants that are born each year worldwide, 15 million are born preterm, increasing their susceptibility to health challenges as a consequence of hindered intrauterine development. Ensuring proper nutrition for these patients is of the utmost importance to allow for normal growth and organ development and overcoming preterm health conditions.

Enteral nutrition is a form of alimentation and metabolic support in which nutrient formulas or medications are delivered directly to the gastrointestinal tract, through either the stomach, duodenum or jejunum. Nutrient administration is performed using an enteral feeding system that includes an enteral feeding container suspended above patient level and a length of flexible administration tubing connected to the container and connected to an in-dwelling enteral feeding tube. Fluid nutrient flows through the enteral feeding tube via either gravity feed, syringe bolus or use of an enteral feeding pump. Excessive gastric pressure can result during feeding, for example through accumulation of gas or liquid resulting from stomach contractions, movement of the patient's abdomen, crying or through normal formation of gas. Typically, the body relieves such excess gastric pressure by expelling accumulated gas or liquid through a burping response. However, in enteral feeding where fluid nutrients are continually fed to the gastrointestinal tract, upward expulsion of gastric reflux materials is highly undesirable. Additionally, certain medical conditions prevent or limit the body's ability to eructate. Because gastric reflux pressure cannot overcome the greater forward fluid pressure within the enteral feeding tube, gastric pressure relief (also referred to as gastric decompression) devices have been developed to relieve gastric reflux pressure through the enteral feeding tube to avoid uncontrolled upward expulsion of reflux materials through the burping response; such devices also prevent introduction of air into any portion of the enteral feeding system, particularly the enteral feeding tube.

Furthermore, patients requiring enteral feeding may further require additional intensive medical interventions. As an example, many neonatal patients require respiratory assistance via continuous positive airway pressure (CPAP). During CPAP treatment, a condition sometimes known as aerophagia may occur. For instance, aerophagia can occur when CPAP pressure is higher than required for the patient, thus extra air has nowhere else to go and is directed into the esophagus and belly, or lower than required for the patient, and the patient gulps in an effort to try to get more air into the lungs. As a result of air being swallowed into the digestive system, the patient may experience abdominal distention and require relief from excess gas or air trapped in the digestive system.

Currently, air venting systems for neonatal patients are insufficient to provide venting or relief of abdominal distention while maintaining a closed system. For instance, a syringe may be coupled to an enteral extension set and left open (e.g., without inserting the plunger, or inserting a plunger only to a vented zone of the syringe) to enable gas and/or residual feed to vent out through the extension set into the syringe. Because the syringe is either left entirely open or at least partially open through a vented section, this existing venting workaround may permit ingress of foreign substances through the syringe and ultimately into the patient, increasing the risk of contamination or illness to the vulnerable neonatal patient.

Consequently, there is a need for improved systems, apparatus and methods for gastric pressure relief and relief of abdominal distention. In particular, a closed system for gastric pressure relief and relief of abdominal distention which prevents ingress of foreign substances would be advantageous.

### SUMMARY

Objects and advantages of the invention will be set forth in part in the following description, or may be obvious from the description, or may be learned through practice of the invention.

The present invention is directed to a gastric decompression apparatus. The apparatus includes a variable-volume container defining an interior volume. The variable-volume container has a containment barrel and a movable plunger slidably mounted within the containment barrel so as to vary the contained volume of the container as the movable plunger slides within the containment barrel. The container includes a coupling at a first end of the container opposite the movable plunger, the coupling having a fluid channel extending therethrough configured to permit fluid ingress or egress from the containment barrel. The movable plunger has a plunger body extending from a first end to a second end. The plunger body has a plunger face at the first end, a plunger head at the second end, and an elongate portion extending therebetween. There is at least one lumen extending through the plunger face, the elongate portion, and the plunger head, and a filter disposed within the lumen.

In one embodiment of the gastric decompression apparatus, the filter may be a gas-permeable filter.

In another embodiment, the filter may be a hydrophobic filter.

In another embodiment, the plunger face may include an opening in communication with the at least one lumen, the filter being disposed in the opening.

In another embodiment, the containment barrel of the variable-volume container may have a length extending from a first end to a second end thereof, the second end receiving the movable plunger, the length being in a range from about 30 millimeters to about 150 millimeters.

In another embodiment, the variable-volume container may have a maximum length defined from a first end of the containment barrel to the plunger head when the plunger is mounted within the containment barrel, the maximum length being in a range from about 35 millimeters to about 350 millimeters.

In another embodiment, the containment barrel of the variable-volume container may have an outer diameter in a range from about 10 millimeters to about 75 millimeters.

In another embodiment, the containment barrel may have a length extending from a first end to a second end thereof and an outer diameter, wherein a ratio of the length to the outer diameter may be in a range from about 1:1 to about 5:1.

In another embodiment, the variable-volume container may have a maximum volume of about 100 milliliters.

In another embodiment, the apparatus may further include a hanger extending from the plunger head.

The present invention further includes a system for gastric decompression. The system includes a venting apparatus comprising a variable-volume container defining an interior volume having a containment barrel and having a movable plunger slidably mounted within the containment barrel so as to vary the contained volume of the container as the movable plunger slides within the containment barrel; the container comprising a coupling at a first end of the container opposite the movable plunger, the coupling having a fluid channel extending therethrough configured to permit fluid ingress or egress from the containment barrel; the movable plunger having a plunger body extending from a first end to a second end, the plunger body having a plunger face at the first end, a plunger head at the second end, and an elongate portion extending therebetween; at least one lumen extending through the plunger face, the elongate portion, and the plunger head; and a filter disposed within the lumen. The system further includes a tube extending from a proximal end to a distal end, the proximal end having a connector configured to be coupled with the coupling of the container, the distal end configured to be inserted into a patient's gastric system such that gas may be vented from the patient's gastric system through the tube, the container, and the lumen of the movable plunger.

In one embodiment of the system, the filter may be a hydrophobic filter.

In another embodiment, the plunger face may include an opening in communication with the at least one lumen, the filter being disposed in the opening.

In another embodiment, the variable-volume container may have a maximum length defined from a first end of the containment barrel to the plunger head when the plunger is mounted within the containment barrel, the maximum length being in a range from about 35 millimeters to about 350 millimeters.

In another embodiment, the containment barrel of the variable-volume container may have an outer diameter in a range from about 10 millimeters to about 75 millimeters.

In another embodiment, the containment barrel may have a length extending from a first end to a second end thereof and an outer diameter, wherein a ratio of the length to the outer diameter may be in a range from about 1:1 to about 5:1.

The present invention further includes a method for gastric decompression. The method includes comprising steps of: providing a gastric decompression system; connecting the coupling with a connector of an enteral feeding tube, the enteral feeding tube having the connector at a proximal end thereof and a distal end disposed within a patient's stomach; venting contents of the patient's stomach into the container; and venting gas from the container through the lumen of the movable plunger. The system includes a venting apparatus comprising a variable-volume container defining an interior volume having a containment barrel and having a movable plunger slidably mounted within the containment barrel so as to vary the contained volume of the container as the movable plunger slides within the containment barrel; the container comprising a coupling at a first end of the container opposite the movable plunger, the coupling having a fluid lumen extending therethrough configured to permit fluid ingress or egress from the containment barrel; the movable plunger having a plunger body extending from a first end to a second end, the plunger body having a plunger face at the first end, a plunger head at the second end, and an elongate portion extending therebetween; at least one lumen extending through the plunger face, the elongate portion, and the plunger head; and a filter disposed within the lumen.

In one embodiment, the method may further include a step of delivering contents of the container to a patient's stomach.

In another embodiment, the step of delivering contents of the container to a patient's stomach may include: covering the lumen on the plunger head; and moving the movable plunger in a direction of the first end of the container such that contents of the container are pushed through the fluid channel of the container into the enteral feeding tube.

In another embodiment, the method may further include a step of collecting contents of a patient's stomach within the container by covering the lumen on the plunger head; and moving the movable plunger in a direction of the second end of the container such that contents of the patient's stomach are suctioned through the enteral feeding tube and through the fluid channel into the container.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a perspective view of a gastric decompression apparatus according to an embodiment of the present invention;

FIG. 2 illustrates a side cross-sectional view of a variable-volume container of the gastric decompression apparatus FIG. 1;

FIG. 3 illustrates a perspective view of a plunger of the gastric decompression apparatus FIG. 1;

FIG. 4 illustrates a side view of the plunger of FIG. 3;

FIG. 5 illustrates a cross-sectional view of the plunger of FIG. 3;

FIG. 6 illustrates a bottom view of the plunger of FIG. 3;

FIG. 7 illustrates a gastric decompression system according to an embodiment of the present invention;

FIG. 8 illustrates the gastric decompression system venting gases according to an embodiment of the present invention; and

FIG. 9 illustrates the gastric decompression system being used as a syringe to deliver residual feed according to an embodiment of the present invention.

### DETAILED DESCRIPTION

Reference now will be made in detail to embodiments of the present invention, one or more examples of which are illustrated in the drawings. The word “exemplary” is used herein to mean “serving as an example, instance, or illustration.” Any implementation described herein as “exemplary” is not necessarily to be construed as preferred or advantageous over other implementations. Moreover, each example is provided by way of explanation, rather than limitation of, the technology. In fact, it will be apparent to those skilled in the art that modifications and variations can be made in the present technology without departing from the scope or spirit of the claimed technology. For instance, features illustrated or described as part of one embodiment can be used with another embodiment to yield a still further embodiment. Thus, it is intended that the present disclosure covers such modifications and variations as come within the scope of the appended claims and their equivalents. The detailed description uses numerical and letter designations to refer to features in the drawings. Like or similar designations in the drawings and description have been used to refer to like or similar parts of the invention.

As used herein, the terms “first”, “second”, and “third” may be used interchangeably to distinguish one component from another and are not intended to signify location or importance of the individual components. The singular forms “a,” “an,” and “the” include plural references unless the context clearly dictates otherwise. The terms “coupled,” “fixed,” “attached to,” and the like refer to both direct coupling, fixing, or attaching, as well as indirect coupling, fixing, or attaching through one or more intermediate components or features, unless otherwise specified herein. As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive- or and not to an exclusive- or. For example, a condition A or B is satisfied by any one of the following: A is true (or present) and B is false (or not present), A is false (or not present) and B is true (or present), and both A and B are true (or present).

Terms of approximation, such as “about,” “generally,” “approximately,” or “substantially,” include values within ten percent greater or less than the stated value. When used in the context of an angle or direction, such terms include within ten degrees greater or less than the stated angle or direction. For example, “generally vertical” includes directions within ten degrees of vertical in any direction, e.g., clockwise or counter-clockwise.

Benefits, other advantages, and solutions to problems are described below with regard to specific embodiments. However, the benefits, advantages, solutions to problems, and any feature(s) that may cause any benefit, advantage, or solution to occur or become more pronounced are not to be construed as a critical, required, or essential feature of any or all the claims.

In general, the present invention is directed to a gastric decompression apparatus includes a variable-volume container having a movable plunger. The movable plunger is slidably mounted within the containment barrel so as to vary the contained volume of the container. The movable plunger has a plunger body having at least one lumen extending therethrough, and a filter disposed within the lumen. The present invention is further directed to a gastric decompression system includes the gastric decompression apparatus and a catheter coupled to the container and a method of providing gastric decompression using the gastric decompression system. The present inventors have found that the gastric decompression apparatus, system, and method of the present invention provides an easy-to-use solution for venting gases from abdominal distention and collecting reflux that further provides capability to “re-feed” reflux contents or residual feed to a patient without the need for additional vents or devices. Additionally, the gastric decompression apparatus may have a larger diameter and a shorter length from the first end to the second end as compared to conventional elongated syringes having an equal volume to minimize the size of apparatus within an isolette for a neonatal patient, advantageously reducing crowding within the isolette, particularly when there may be numerous tubes, wires, and monitors present for treating the neonatal patient.

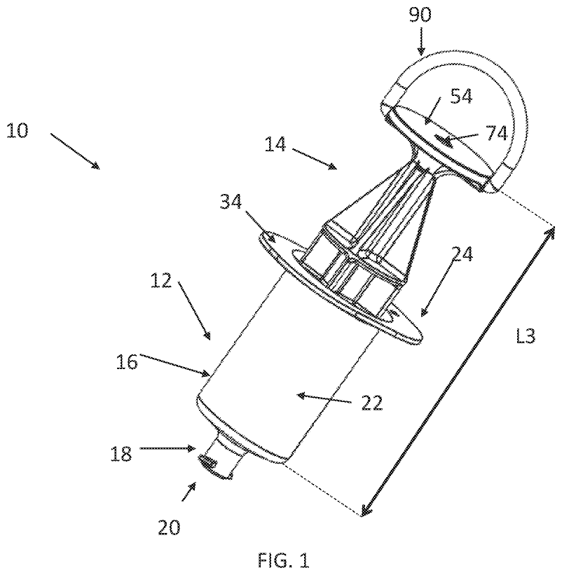

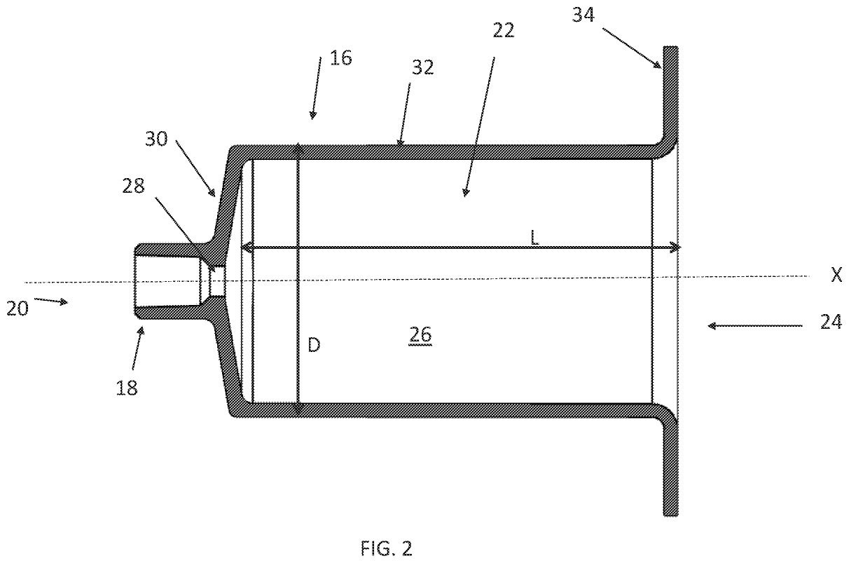

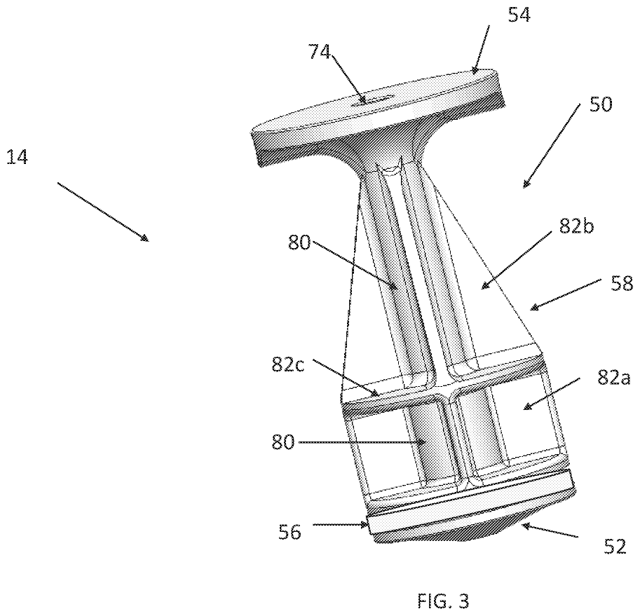

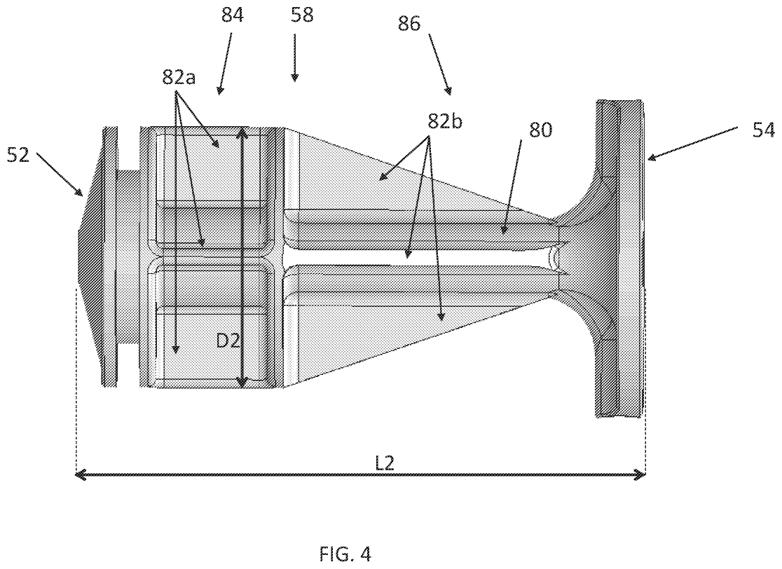

Referring now to the drawings, FIG. 1 illustrates a gastric decompression apparatus 10. The gastric decompression apparatus 10 includes a variable-volume container 12 and a movable plunger 14. For instance, the variable-volume container 12 and movable plunger 14 may be in the form of a syringe configured to contain fluids, e.g., human breast milk, nutritional formula, liquid medicine, or any other desired fluid for delivery to a patient via enteral feeding. The variable-volume container 12 includes a body 16 having a coupling 18 at a first end 20 and a containment barrel 22 extending from the coupling 18 to a second end 24 of the body 16. The containment barrel 22 surrounds a bore 26 of the variable-volume container 12 which forms a contained volume. The plunger 14 may be slidably mounted within the containment barrel 22 (i.e., the plunger 14 may slide back and forth within the containment barrel 22 and/or may be slidably inserted into or removed from the containment barrel 22).

The containment barrel 22, sometimes referred to as a syringe barrel, has a radial base 30 extending in a radial direction from a central axis X of the body 16 and a sidewall 32 surrounding the bore 26. Optionally, the body 16 may have a flange 34 extending radially from the sidewall 32 of the containment barrel 22 on an opposite end relative to the radial base 30. The flange 34 may provide a gripping surface or handling surface when the plunger is being moved relative to the bore 26.

The containment barrel 22 is in fluid communication with the coupling 18 by a lumen or channel 28. The channel 28 extends through the coupling 18 from the first end 20 to the radial base 30 of the containment barrel 22 and extends through the radial base 30 to be fluidly coupled with the bore 26. The channel 28 may permit fluid ingress or egress from the containment barrel 22.

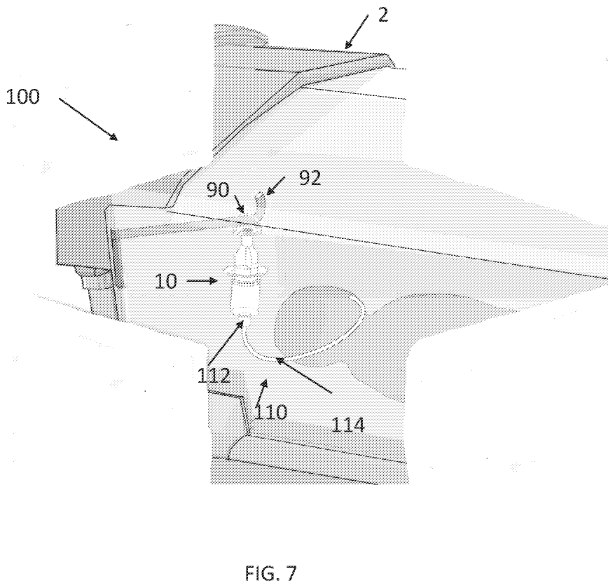

The coupling 18, including the channel 28, may be configured to couple with a connector 112 of a catheter 110, such as an enteral feeding tube (described in further detail below). For instance, the coupling 18 may be configured to couple to an enteral feeding tube of a patient (as illustrated in FIG. 7). In some aspects, the coupling 18 may comply with the ISO 80369-3 standard for connectors for enteral applications, commonly referred to as ENFIT.

Still referring to FIG. 1, the movable plunger 14 has a plunger body 50 extending from a plunger face 52 at a first end to a plunger head 54 at a second end. The plunger face 52 has an outer diameter that is equal to or slightly greater than the diameter of the bore 26 such that the plunger face 52 may be inserted into the bore 26. The plunger face 52 may include a gasket 56 which comprises an annular band or ring extending around the diameter of the plunger face 52. The gasket 56 may be fabricated from a resilient material providing a good seal and a low coefficient of friction with the interior surface of the containment barrel 22, such as for example a silicone rubber elastomer. The plunger face 52 may also be formed from the same material as the gasket 56. For instance, the plunger face 52 and the gasket 56 may be integrally formed from a single piece of material.

The plunger 14 further includes a filter 60. The filter 60 may be disposed between an elongate portion 58 of the body 50 and the plunger face 52. The filter 60 may be a gas-permeable filter. Further, the filter 60 may be a hydrophobic filter. For instance, in some aspects, the filter 60 may be liquid-impermeable. In some aspects of the present invention, the filter 60 may have a filter body 62 and a piece of filter media 64. The filter body 62 may hold the piece of filter media 64 in place. For instance, the filter body 62 may surround the piece of filter media 64. In some aspects, the filter body 62 may have a generally circular cross-sectional shape. The filter body 62 may be inserted and/or held in place relative to the plunger body 50, as will be described in further detail below.

The plunger body 50 has a lumen 70 extending therethrough. The lumen 70 may extend from the plunger face 52 to the plunger head 54. In some aspects, the lumen may extend concentric with the central axis X. The lumen 70 is surrounded by a lumen outer wall 72. The lumen outer wall 72 may be integrally formed with the plunger body 50. For instance, the lumen outer wall 72 may be integrally molded with the plunger body 50.

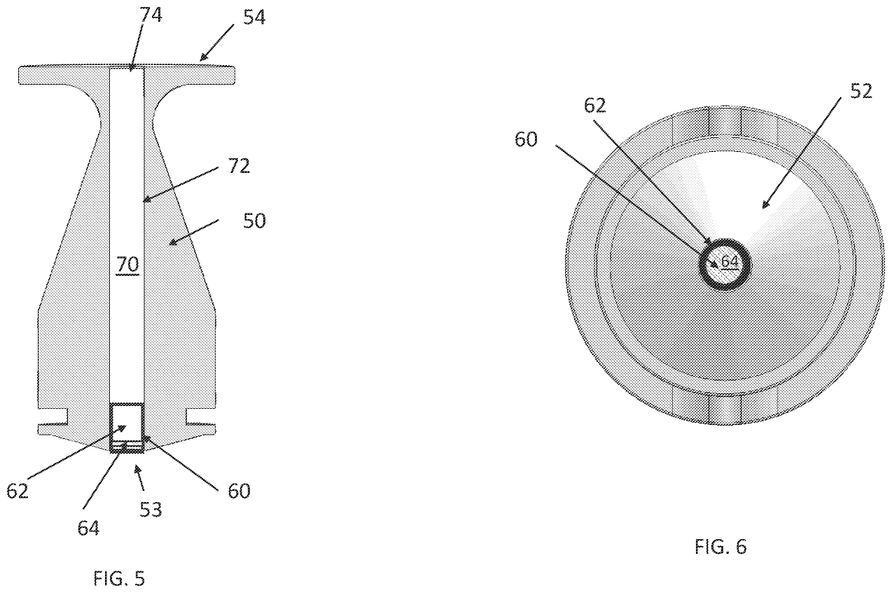

As illustrated in FIG. 5-6, the plunger face 52 includes an opening 53 providing access to the lumen 70. For instance, the opening 53 may be generally centrally disposed on the plunger face 52. In some aspects, the opening 53 may be concentric with the central axis X. At an opposite end of the lumen 70, the plunger head 54 includes an opening 74 providing access to the lumen 70.

The filter 60 may be disposed within the lumen 70 of the plunger 14. In this regard, the lumen 70 of the plunger 14 may be generally permeable to gas and hydrophobic or impermeable to liquid. For instance, the filter 60 may be disposed near or adjacent to the plunger face 52, the plunger head 54, or any suitable position within the lumen 70. In some aspects of the invention, the plunger body 50 may have a recess 76 configured to receive the filter body 62, e.g., at the first end or second end thereof. The filter body 62 may be inserted into the recess 76. In some aspects, the recess 76 may be coaxial with the lumen 70. In this regard, the filter media 64 may cover access to the lumen 70, e.g., at the first end of the plunger body 50 as illustrated in FIGS. 5-6. The filter body 62 may be held in place relative to the plunger body 50 by an interference fit. Additionally or alternatively, the filter body 62 may be bonded in place relative to the plunger body 50 such as by adhesive or other suitable bonding methods. During formation of the plunger 14, the filter body 62 may be inserted relative to the plunger body 50 and the gasket 56 of the plunger face 52 may then be coupled to the plunger body 50. In this arrangement, the opening 53 of the gasket 56 may expose the filter media 64 therethrough.

The elongate portion 58 of the plunger body 50 may be formed with a central body portion 80 surrounding the lumen 70 and the lumen outer wall 72, and a plurality of ribs 82 radially extending from the central body portion 80. For instance, in one aspect of the invention, there may be a plurality of ribs 82 radially spaced apart about 90 degrees. The elongate portion 58 may have a generally cylindrical section 84 and a generally conical section 86. The generally cylindrical section 84 may be disposed adjacent to the plunger face 52. The ribs 82a in the generally cylindrical section 84 may extend to the outer diameter D2 of the plunger 50. For instance, the ribs 82a in the generally cylindrical section 84 may have a generally rectangular shape. In contrast, the ribs 82b in the generally conical section 86 may taper from the outer diameter D2 to the central body portion 80. The ribs 82b in the generally conical section 86 may have a generally triangular shape. A circular rib may extend radially from the central body portion 80 to the outer diameter D2

The plunger 14 may further include a hanger 90 configured to hang the plunger 14 from a hanging element 92 such as a rod or hook (see FIG. 7). For example, when the gastric decompression apparatus 10 is used for a neonatal patient, the hanger 90 can be hung from a hook within an isolette 2, as shown in FIG. 5. The hanger 90 may extend from the plunger body 50, e.g., at or near the second end of the plunger body 50 so that the hanger 90 does not impede insertion of the plunger body 50 into the container barrel 22. For example, the hanger 90 may extend from the plunger head 54. The hanger 90 may be integrally formed with the plunger body 50, such as by integrally molding the hanger with the plunger body 50, or may be formed separately and coupled to the plunger body 50. In some aspects, the hanger 90 may be formed from a flexible, pliable material such that the hanger 90 may conform to various types of hanging elements. However, any suitable means for hanging or mounting the gastric decompression apparatus 10 in or on an isolette for a neonatal patient may be used with the present invention.

Additionally, the present invention contemplates a unique size for the gastric decompression apparatus 10. In particular, the gastric decompression apparatus 10 may have a larger diameter and a shorter length from the first end to the second end as compared to conventional elongated syringes having an equal volume. Specifically, the barrel 22 may have an outer diameter D and a length L between the coupling 18 and the second end 24. For instance, a ratio of L:D of the containment barrel 22 may be in a range from about 1:1 to about 5:1, such as from about 1:1 to about 3.5:1, such as from about 1.5:1 to about 3:1. By providing a gastric decompression apparatus 10, and specifically a containment barrel 22, having a shorter length L, and in turn a larger diameter D to accommodate a desired volume, the gastric decompression apparatus 10 may fit more easily within an isolette for a neonatal patient and take up less space in the isolette than a conventional syringe. Such a distinction between the gastric decompression apparatus 10 of the present invention and conventional syringes is not trivial, as the present inventors have found that minimizing the size of the syringe within an isolette for a neonatal patient advantageously reduces crowding within the isolette, particularly when there may be numerous tubes, wires, and monitors present for treating the neonatal patient.

The diameter D of the containment barrel 22 may be in a range from about 10 millimeters to about 75 millimeters, such as from about 15 to about 50 millimeters, for instance, from about 25 to about 35 millimeters.

The length L of the containment barrel 22 may be in a range from about 20 millimeters to about 150 millimeters, such as from about 30 to about 100 millimeters, for instance, from about 40 to about 60 millimeters.

The plunger body 50 has a plunger length L2 extending from the plunger face 52 to the plunger head 54. The length L2 may be in a range from about 30 millimeters to about 180 millimeters, such as from about 40 to about 150 millimeters, for instance, from about 45 to about 100 millimeters.

The variable-volume container 12 may have a maximum length L3 defined from the radial base of the container 12 to the plunger head 54 in a configuration when the plunger 14 is withdrawn within the bore 26 towards the second end 24 of the body 16 while the plunger face 52 remains within the bore 26 and the gasket 56 remains sealingly engaged with the outer wall 32 of the container body 22. The maximum length L3 may be in a range from about 35 millimeters to about 350 millimeters, such as from about 50 millimeters to about 200 millimeters, for instance from about 75 millimeters to 140 millimeters.

The variable-volume container 12 may have a maximum interior volume of about 200 milliliters, such as a maximum volume of about 100 milliliters, such as a maximum volume of about 60 milliliters, such as a maximum volume of about 20 milliliters. The variable-volume container 12 may have a volume in a range from about 0 milliliters when the plunger 14 is fully inserted into the bore 26 toward the first end 20 of the body 16, up to the maximum volume when the plunger 14 is withdrawn within the bore 26 towards the second end 24 of the body 16 while the plunger face 52 remains within the bore 26 and the gasket 56 remains sealingly engaged with the outer wall 32 of the container body 22.

In one particular embodiment, the maximum length L3 may be in a range from about 95 millimeters to about 135 millimeters and the container 12 may have a maximum interior volume of about 20 milliliters.

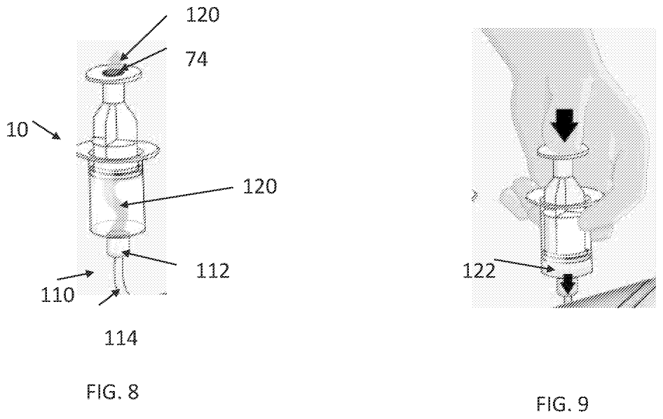

A gastric decompression system 100 includes the gastric decompression apparatus 10 and a catheter 110, such as nasogastric feeding tube or other feeding tube, as illustrated in FIG. 7-8. The catheter 110 may include a connector 112 at a proximal end of a catheter tube body 114 and an opening (not shown) at a distal end of the catheter tube body 114. The catheter tube body 114 may be configured to be inserted into a patient's body, e.g., into a patient's digestive tract, so that the opening 116 is disposed in the digestive tract, for instance in the patient's stomach.

In an assembled configuration of the gastric decompression system 100, the plunger face 52 of the plunger 14 is disposed within the barrel 22, and the coupling 18 is connected with a connector 112 of the catheter 110. In this configuration, contents 120, 122 within the catheter 110 (e.g., residual feed, such as by reflux), can be vented into the barrel 22 by flowing through the catheter tube body 114 and the channel 28 into the bore 26. Moreover, any gases 120 received within the bore 26 may be vented through the lumen 70 of the plunger 14. Due to the hydrophobic properties of the filter media 64, any liquid contents 122 within the barrel 22 or bore 26 may be contained and not spilled out through the lumen 70 of the plunger 14.

In some aspects of the present invention, the gastric decompression system 100 may provide gastric relief from abdominal distention caused by ventilation with continuous positive airway pressure (CPAP). For instance, the gastric decompression apparatus 10 may be connected with a nasogastric tube 110 at any time when the patient is not actively receiving a feed through the nasogastric tube 110. The gastric decompression apparatus 10 then provides a means for venting gases from the patient's stomach or digestive system, thereby relieving potential abdominal distention.

Additionally, the gastric decompression system 100 may be used to deliver and/or remove contents from a patient's digestive system (see FIG. 9). When the opening 74 on the plunger head 54 is covered or sealed, e.g., by a user's finger, gas may be inhibited from venting and the gastric decompression apparatus 10 may function as a syringe. Thus, when the opening 74 is covered or sealed, the plunger 14 may be depressed toward the coupling 18 of the container 12 to push and deliver the contents within the bore 26 back through the catheter 110 and into the patient's body. For example, if any stomach contents such as residual feed are collected within the container 12 as a result of reflux, the stomach contents can be re-delivered (or “re-fed”) to the patient by implementing the gastric decompression apparatus 10 as a syringe for delivering nutrition. Conversely, when the opening 74 is covered or sealed, the plunger 14 may also be pulled away from the coupling 18 of the container 12 to suction contents from the patient's body through the catheter 110 and into the bore 26 of the container 12.

In particular, the present invention includes a method for gastric decompression implementing the gastric decompression system 100 as described above. The method may be performed when a feeding tube is already in place in a patient's stomach. After coupling the gastric decompression apparatus 10 to the feeding tube, e.g., nasogastric tube 110, the method includes a step of venting contents of the patient's stomach into the container 12. Specifically, because the container 12 enables gas to vent out through the filter 60 and opening 74 of the plunger 14, the pressure within the bore 26 is generally equal to the environmental pressure and thus does not force gas into nor suction gas from the nasogastric tube 110. This enables contents of the patient's stomach, including gas from abdominal distention during CPAP and/or residual feed from a reflux event, to be collected in the container 12. The method further includes a step of venting gas from the container 12 out through the lumen 70 of the movable plunger 14.

In a scenario in which residual feed 122 has collected in the container 12, e.g., due to reflux, the method may further include a step of delivering contents of the container to a patient's stomach. Specifically, the opening 74 on the plunger head 54 may be covered or sealed so that gas may not be vented, and the plunger 14 may be pushed toward the first end 20 of the container 12 to push the contents 122 back into the nasogastric tube 110.

In a similar manner, the gastric decompression apparatus 10 may be used to collect contents 120, 122 of a patient's stomach. The, the opening 74 on the plunger head 54 may be covered or sealed so that gas may not be vented, and the plunger 14 may be pulled toward the second end 24 of the container, i.e., away from the nasogastric tube 110, to collect gaseous and/or liquid contents from a patient's stomach.

While the gastric decompression apparatus 10 and decompression system 100 have been described above specifically with regard to enteral feeding and relief of abdominal distention from enteral feeding and respiratory treatment via CPAP, the present inventors contemplate that the gastric decompression apparatus 10 and decompression system 100 can be used in a variety of clinical and non-clinical applications or any other desirable use for which the present invention may be deemed appropriate. For example, the gastric decompression apparatus 10 could be used to function as a gravity feed solution, i.e., to deliver nutrition to a patient.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims.

1. A gastric decompression apparatus comprising:
a variable-volume container defining an interior volume having a containment barrel and having a movable plunger slidably mounted within the containment barrel so as to vary the contained volume of the container as the movable plunger slides within the containment barrel;
the container comprising a coupling at a first end of the container opposite the movable plunger, the coupling having a fluid channel extending therethrough configured to permit fluid ingress or egress from the containment barrel;
the movable plunger having a plunger body extending from a first end to a second end, the plunger body having a plunger face at the first end, a plunger head at the second end, and an elongate portion extending therebetween;
at least one lumen extending through the plunger face, the elongate portion, and the plunger head; and
a filter disposed within the lumen.

2. The gastric decompression apparatus of claim 1, wherein the filter is a gas-permeable filter.

3. The gastric decompression apparatus of claim 1, wherein the filter is a hydrophobic filter.

4. The gastric decompression apparatus of claim 1, the plunger face comprising an opening in communication with the at least one lumen, the filter being disposed in the opening.

5. The gastric decompression apparatus of claim 1, the containment barrel of the variable-volume container having a length extending from a first end to a second end thereof, the second end receiving the movable plunger, the length being in a range from about 30 millimeters to about 160 millimeters.

6. The gastric decompression apparatus of claim 1, the variable-volume container having a maximum length defined from a first end of the containment barrel to the plunger head when the plunger is mounted within the containment barrel, the maximum length being in a range from about 35 millimeters to about 350 millimeters.

7. The gastric decompression apparatus of claim 1, the containment barrel of the variable-volume container having an outer diameter in a range from about 10 millimeters to about 75 millimeters.

8. The gastric decompression apparatus of claim 1, the containment barrel having a length extending from a first end to a second end thereof and an outer diameter, wherein a ratio of the length to the outer diameter is in a range from about 1:1 to about 5:1.

9. The gastric decompression apparatus of claim 1, the variable-volume container having a maximum volume of about 100 milliliters.

10. The gastric decompression apparatus of claim 1, further including a hanger extending from the plunger head.

11. A system for gastric decompression comprising:
a venting apparatus comprising a variable-volume container defining an interior volume having a containment barrel and having a movable plunger slidably mounted within the containment barrel so as to vary the contained volume of the container as the movable plunger slides within the containment barrel;
the container comprising a coupling at a first end of the container opposite the movable plunger, the coupling having a fluid channel extending therethrough configured to permit fluid ingress or egress from the containment barrel;
the movable plunger having a plunger body extending from a first end to a second end, the plunger body having a plunger face at the first end, a plunger head at the second end, and an elongate portion extending therebetween;
at least one lumen extending through the plunger face, the elongate portion, and the plunger head; and
a filter disposed within the lumen; and
a tube extending from a proximal end to a distal end, the proximal end having a connector configured to be coupled with the coupling of the container, the distal end configured to be inserted into a patient's gastric system such that gas may be vented from the patient's gastric system through the tube, the container, and the lumen of the movable plunger.

12. The gastric decompression system of claim 11, wherein the filter is a hydrophobic filter.

13. The gastric decompression system of claim 11, the plunger face comprising an opening in communication with the at least one lumen, the filter being disposed in the opening.

14. The gastric decompression system of claim 11, the variable-volume container having a maximum length defined from a first end of the containment barrel to the plunger head when the plunger is mounted within the containment barrel, the maximum length being in a range from about 35 millimeters to about 350 millimeters.

15. The gastric decompression system of claim 11, the containment barrel of the variable-volume container having an outer diameter in a range from about 10 millimeters to about 75 millimeters.

16. The gastric decompression system of claim 11, the containment barrel having a length extending from a first end to a second end thereof and an outer diameter, wherein a ratio of the length to the outer diameter is in a range from about 1:1 to about 5:1.

17. A method for gastric decompression comprising steps of:
providing a gastric decompression system, the gastric decompression system comprising:
a venting apparatus comprising a variable-volume container defining an interior volume having a containment barrel and having a movable plunger slidably mounted within the containment barrel so as to vary the contained volume of the container as the movable plunger slides within the containment barrel;
the container comprising a coupling at a first end of the container opposite the movable plunger, the coupling having a fluid lumen extending therethrough configured to permit fluid ingress or egress from the containment barrel;
the movable plunger having a plunger body extending from a first end to a second end, the plunger body having a plunger face at the first end, a plunger head at the second end, and an elongate portion extending therebetween;
at least one lumen extending through the plunger face, the elongate portion, and the plunger head; and
a filter disposed within the lumen;
connecting the coupling with a connector of an enteral feeding tube, the enteral feeding tube having the connector at a proximal end thereof and a distal end disposed within a patient's stomach;
venting contents of the patient's stomach into the container, and
venting gas from the container through the lumen of the movable plunger.

18. The method of claim 17, further comprising a step of delivering contents of the container to a patient's stomach.

19. The method of claim 18 wherein the step of delivering contents of the container to a patient's stomach comprises:
covering the lumen on the plunger head; and
moving the movable plunger in a direction of the first end of the container such that contents of the container are pushed through the fluid channel of the container into the enteral feeding tube.

20. The method of claim 17, further comprising a step of collecting contents of a patient's stomach within the container by covering the lumen on the plunger head; and
moving the movable plunger in a direction of the second end of the container such that contents of the patient's stomach are suctioned through the enteral feeding tube and through the fluid channel into the container.
